(19) United States
(12) Patent Application Publication
(10) Pub. No.: US 2024/0003869 A1
(43) Pub. Date: Jan. 4, 2024

(54) PLASMONIC CELL MASS ACCUMULATION PROFILING PLATFORM FOR DETERMINING THERAPEUTIC RESPONSE OF CANCER CELLS

(71) Applicant: IZMIR BIYOTIP VE GENOM MERKEZI, Izmir (TR)

(72) Inventor: Arif Engin CETIN, Izmir (TR)

(73) Assignee: IZMIR BIYOTIP VE GENOM MERKEZI, Izmir (TR)

(21) Appl. No.: 18/255,377

(22) PCT Filed: Nov. 10, 2021
(86) PCT No.: PCT/TR2021/051179

§ 371 Date: Jun. 1, 2023

(30) Foreign Application Priority Data
Dec. 2, 2020 (TR) 2020/19537

(51) Int. Cl. G01N 33/50; G01N 21/552

(52) U.S. Cl. CPC G01N 33/5011; G01N 21/554

(57) ABSTRACT

A plasmonic-based biosensor platform that determines the biophysical properties of cells, the changes within, and their therapeutic behavior upon the molecules that cause these changes in an ex vivo and label-free manner is provided. The plasmonic-based biosensor platform includes a plasmonic chip, a light source, an inverted microscope, an incubator case, an optical read-out device, and a graphical user interface. The biosensor platform of the invention could determine the therapeutic susceptibility of cancer cells to cancer drugs in a label-free manner.

### CROSS REFERENCE TO THE REPLATED APPLICATIONS

This application is the national phase entry of International Application No. PCT/TR2021/051179, filed on Nov. 10, 2021, which is based upon and claims priority to Turkish Patent Application No. 2020/19537, filed on Dec. 2, 2020, the entire contents of which are incorporated herein by reference.

### TECHNICAL FIELD

The invention relates to a plasmonic-based in-vitro functional analysis platform that determines the therapeutic responses of cancer models with single-cell sensitivity.

### BACKGROUND

In recent years, in parallel with the scientific and technological developments in the field of medicine, with the increase in the options for the treatment of cancer, many cancer patients overcame the disease, and their life quality improved. However, it is difficult to completely eradicate this disease. The most important reason for this difficulty is the resistance of cancer cells to drugs used in cancer treatment. For this reason, it is of great importance to determine a rapid and accurate personalized drug therapy for cancer treatment.

Label-free optical biosensing platforms eliminated the need for optical labels (e.g., fluorescent dyes) for detection with the use of special electromagnetic waves called surface plasmons. Sensing variety of bio-targets has been successfully demonstrated, from small biomolecules (e.g., protein, Masson and Zhao 2015) to large organisms (e.g., bacteria or virus, Massad-Ivanir et al. 2013).

This labeled optical method, which is successful in identifying different bio-targets, has not been used to investigate the biophysical properties of cells or to determine their therapeutic behavior yet. In state of art, instead of product or application-oriented studies, there is basic research on optical, chemical and biological methods for labeled cell-based biosensing technologies. In some of these studies, cells were not directly used in biosensing platforms, rather they aimed to identify molecules (e.g., intracellular or extracellular proteins) involved in cellular pathways. For example, Eletxigerra et al. (2016) realized the detection of ErbB2, an epidermal growth factor receptor involved in cell proliferation, growth, apoptosis and differentiation, and is associated with cellular signaling pathways, using a gold nanoparticle-based surface plasmon resonance (SPR) system, and performed successful quantitative analyzes based on monitoring of SPR signals.

In literature, there are studies aimed to test plasmonic substrates for cell adherence, and to determine how cellular behaviors are affected with the variations due to the substrates. For example, Giner-Casares et al. (2016) controlled the morphology of human umbilical vein endothelial cells (HUVEC) by functionalizing gold nanoparticles with cyclic argilglycelaspartic acid (c-RGD) peptide. In this platform, cells were successfully separated from the plasmonic surface via a near infrared (NIR) laser, while cell viability was preserved.

Similarly, Tu et al. (2017) studied the real-time cell-substrate interaction dynamics by utilizing microfluidics and plasmonic nanohole geometry, where they showed spectral shifts to longer wavelengths within the transmission response the nanohole geometry as the cells approach the plasmonic surface. Plasmonic structures also enable the investigation of the expression (production and release) levels of molecules and their interaction kinetics with drugs in cells, which are critical for cancer diagnosis and treatment.

Zhang et al. (2015) demonstrated the real-time monitoring of antibody binding to A431 cells with artificially high expression of epidermal growth factor receptor (EGFR, a membrane-bound protein associated with cell survival, proliferation and metabolism). By monitoring SPR signals, the increase in the total biomass due to the antibodies bounding on the cells was detected.

In another study, Li et al. (2017) performed label-free detection of vascular endothelial growth factor (VEGF) with a nanohole-based sensor system. In this system, cells were trapped in a microfluidic circuit, and the biomaterials secreted from the cells were delivered to the nanohole sensors located in another microfluidic chamber. Later, Li et al. (2018) introduced a nanohole -based biosensor that provides the real-time detection of cytokine secretion from cells. In this system, cells were captured on nanohole sensors with a polymer structure called PLL-PEG. The cytokines secreted from cells starting with a chemical stimulus, which were captured by antibodies on the nanohole surface, and this binding event was determined as a spectral change within the transmission response of nanoholes. Goal of these two studies is to characterize single-cell signaling pathways for basic and clinical research.

Therefore, some of these studies mentioned above aim to identify molecules secreted from the cell membrane using the change within the optical responses of the plasmonic structures due to the capture of these molecules by the ligands on the sensor surface, e.g., there is no direct contact with cells. On the other hand, some of these studies examine the adhesion state of the cells on the sensor surface, which varies the optical responses of the plasmonic structure.

Three different technologies are present (which do not utilize plasmonics) that have the potential as an in-vitro functional analysis platform for determining the therapeutic profiles of cells. In the first technology, 2-dimensional imaging of cells is used to calculate increase or decrease in the cell mass via monitoring cell volume (Elfwing et al. 2004, Kim et al. 2018). In this method, the height change in the 3-dimensional cell volume is neglected, and the variation in cell mass is calculated using the change in their area. This weaken the reliability of the method when used in cellular therapeutic profiling based on mass calculation.

In the second technology, a platform was developed to detect changes within the cell volume using atomic force microscopy (AFM) (Van Der Hofstadt et al. 2015). In this technology, the size problem experienced in the imaging technologies was addressed as both diameter and height of cells can be measured. However, the main problem of this method is that the cells need to be scanned in contact with AFM tips. This micro-tip scanning could stress cells, i.e., the measurements may not reliably determine the therapeutic profiles of cells.

The latest technology eliminates the problems associated with these two methods, which is based on the direct measurement of cell mass (Cermak et al. 2016, Stevens et al. 2016). Thus, while adding information coming from the cell height, external factors originating from the measurement method could be eliminated. In this system, cell mass is determined with a mechanical resonator-based diagnostic system, where the cells change the total mass of the resonators by passing over them along a microfluidic chamber integrated to the resonators. Then, the amount of the mass change is determined. By calculating cell mass, the system is able to detect therapeutic profiles with high sensitivity using the mass change information due to the drugs used in the cancer treatments. This system has two main problems. First, cells have to pass over the resonators. For this reason, adherent cell models need to be suspended using suspension protocols. However, the potential of these protocols to stress cells could effect the reliability of therapeutic profiling measurements. The other problem is high-cost of the chips used in this technology due to the need for complex, long and expensive fabrication techniques.

In none of the studies mentioned above, the link between cell mass change and the plasmonic structures of surface covered with cells was investigated. In label-free plasmonic studies in literature, biophysical properties of cells and their therapeutic responses to cancer drugs have not been determined by monitoring cell mass in ex vivo yet.

### SUMMARY

With this invention, a label-free biosensor platform is introduced that can detect cell mass change at single cell sensitivity within 10 minutes, and with a sensitivity of picogram/hour (FIGS. 1A-1C).

The invention is a label-free biosensor platform that can determine the therapeutic effects of drugs or drug combinations at single cell level and with high sensitivity (in the range between 0 and 1 picogram/hour), by the analysis of cell mass accumulation behavior.

The invention determines the changes within the biophysical properties of cells and their therapeutic response against molecules that could possibly cause these changes in a label-free and ex vivo fashion. A single cell is incubated in each sensor region on the surface of the plasmonic chip (2). Changes in the mass of the incubated cells is measured at single cell level. This measurement is either determined by monitoring spectral changes in the transmission response of the nanohole geometry or intensity change in plasmonic diffraction field images.

Another goal of the invention is to develop a label-free biosensor platform that can detect mass accumulation and therapeutic profiles of cell populations with high sensitivity (in the range between 0 and 1 picogram/hour) by monitoring multiple cells all at the same time.

With the invention, a plasmonic-based label-free biosensor platform could be developed for characterization of drug concentration and drug exposure time, and to perform ex vivo functional analyses of therapies developed for cancer patients.

In the invention, by replacing the optical method of spectral tracking of plasmonic modes to that of tracking plasmonic image intensities, throughput capacity of the invention could be increased from 1 cell in 10 minutes to 200-300 cells in 10 minutes (FIG. 5A).

The invention has the potential to be transformed into a device, where the biophysical properties of cells can be investigated for basic research and determining therapeutic behavior of cancer cells enabling the accurate and rapid selection of personalized drug therapy.

The invention detects the change in mass of cancer cells with high sensitivity, in real-time, a short period of time, and label-free manner. A large number of cells belonging to a population are monitored simultaneously to determine the mass accumulation profile of the population. Effects of drug therapies on cells are determined by tracking mass accumulation profile of cells, where the therapeutic response of different cell models are tested all in the same platform. Variations in the mass accumulation profile of cancer models exposed to different drugs are used to determine drug sensitivity or resistance.

With the simultaneous determination of the effects of different cancer drugs without the need for ex vivo cell cultures, drugs that cancer cells are resistant to could be detected, i.e., unnecessary treatment options could be eliminated. This feature enables physicians to make high-accuracy drug therapy selection, which results in successful treatments increasing the survival rate of patients.

The invention has the potential to be utilized in biology and pharmacology such as identifying proteins and cancer biomarkers, and examining their binding dynamics, or detecting pathogens, e.g., bacteria or viruses, which brings new solutions to public health problems.

### BRIEF DESCRIPTION OF THE DRAWINGS

FIG. 1A: High-sensitivity (range 0 to 1 picogram/hour) cell mass accumulation profiling platform. FIG. 1B: Plasmonic sensor chip used in the system. FIG. 1C: Transmission response of the plasmonic chip.

FIG. 2A: Increase in net cell mass due to larger molecular uptake compared to secretion. FIG. 2B: Real-time increase in cell mass shifts the transmission resonance of the plasmonic chip to longer wavelengths. FIG. 2C: The increase in the spectral integral due to the red-shift over time for a cell with increasing mass. FIG. 2D: Decrease in net cell mass due to lower molecular uptake compared to secretion. FIG. 2E: Real-time decrease in cell mass shifts the transmission resonance of the plasmonic chip to shorter wavelengths. FIG. 2F: The decrease in the spectral integral due to the blue-shift over time for a cell with decreasing mass.

FIG. 3A: Calculation of MAR for cells with increasing (top figure) and decreasing (bottom figure) mass in real-time. FIG. 3B: Determining population's MAR vs. mass map by calculating MAR for each cell. FIG. 3C: Normalized MAR profile generated by dividing MAR of each cell with their mass.

FIG. 4A: Investigation of the effects of different cancer drugs with the invention. MAR profile of drug-sensitive cells is negatively affected, while the MAR profile of drug-resistant cells remains constant. The gradual decrease within the population's MAR profile with drug incubation time (FIG. 4B) and concentration (FIG. 4C). FIG. 4D: Effect of single and multiple drug therapies on the MAR profile of the population. For example, Drug-1 and Drug-2 are effective on cancer cells by different amount, and the combination of two drugs (Drug-1+Drug-2) affected the MAR profile of the population more compared to single drug therapies (Drug-1 or Drug-2 only).

FIG. 5A: High-throughput cell mass accumulation profiling platform. FIG. 5B: Working principle of the high-throughput system. FIG. 5C: For a drug-sensitive cell model in the high-throughput system: In the absence of drug therapy, increase in the cell mass increases the plasmonic image intensity, while the drug therapy leads apoptosis such that cells lose mass, decreasing the image intensity.

FIG. 6A: Determination of cell locations seeded on the plasmonic chip in the high-throughput system with the graphical user interface using the camera image. FIG. 6B: Calculation of MAR using the changes within the intensity of the plasmonic image determined with the camera and in real-time. FIG. 6C: Determination of the MAR profile of the population (MAR vs. mass plot), with MAR calculated for each cell. FIG. 6D: Normalized MAR profile generated by dividing MAR by the mass of each cell.

FIG. 7A: Investigation of the effects of different cancer drugs with the invention. MAR profile of the drug-sensitive cells is negatively affected, while the MAR profile of drug-resistant cells remains constant. Determining the effects of (FIG. 7B) drug incubation time and (FIG. 7C) drug concentration on the MAR profile of a population with the use of normalized MAR profile. FIG. 7D: Determination of the effects of different drugs and their combinations from the normalized MAR profile.

FIG. 8: Use of the invention in determining personalized drug treatment for cancer therapy.

### DEFINITIONS OF ELEMENTS/SECTIONS/PARTS OF THE INVENTION

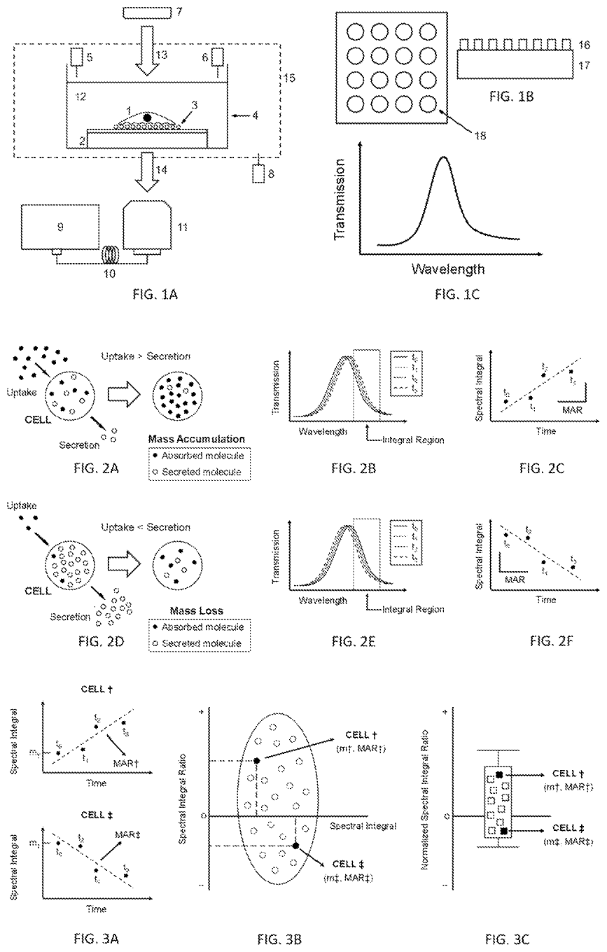

### FIGS. 1A-1C

1: Cell
2: Plasmonic chip
3: Surface modification agent
4: Sample holder
5: CO₂ module
6: Humidity module
7: Light source
8: Temperature module
9: Spectrometer
10: Fiber-coupling optical setup
11: Inverted microscope
12: Cell medium
13: Incoming light
14: Light transmitted from the chip
15: Incubator case
16: Metal film
17: Glass substrate
18: Periodic nanohole array

### FIGS. 5A-5C

19: Camera
20: LC Filter
21: LC control unit
22: Light transmitted from the filter

### DETAILED DESCRIPTION OF THE EMBODIMENTS

The invention relates to a plasmonic biosensor platform that determines the biophysical properties of cells, and their therapeutic response towards molecules that could cause changes in their biophysical properties in a label-free and ex vivo fashion. The biosensor platform of the invention could determine the therapeutic susceptibility of cancer cells to cancer drugs in a label-free way.

The invention of plasmonic biosensor platform includes the following;
A plasmonic chip (2), which consists of periodic nanohole array (18) fabricated on a nm-thick metal film (16), on which the cell to be examined is seeded on its surface,
A light source (7) that illuminates the plasmonic chip (2),
An inverted microscope (11), used to illuminate the plasmonic chip (2), to collect the light transmitted from the plasmonic chip (2), and to send it to the device that performs the optical reading,
An incubator case (15), which provides the necessary incubator conditions for cell culture, integrated to the inverted microscope (11)
An optical read-out device that measures the transmission response of the plasmonic chip (2), which is integrated into the microscope (11),
A graphical user interface that controls the optical read-out device, and employs algorithms to convert optical data to MAR information.

The invention shown in FIG. 1A is the version of the plasmonic biosensor platform which contains;
Plasmonic chip (2), which consists of periodic nanohole array (18) fabricated on a nm-thick metal film (16), of surface where the cell to be examined is seed on,
Light source (7) that illuminates the plasmonic chip (2),
Inverted microscope (11), used to stimulate the optical response of the nanohole array by illuminating the plasmonic chip (2), to collect the light passing through the plasmonic chip, and to send it to the spectrometer (9) performing the optical readings,
Incubator case (15) that provides the conditions suitable for cell culture (Example: 5% CO₂, 37% Temperature, 95% Humidity), contains a CO₂ module (5), a humidity module (6) and a heat module (8), which is integrated to the inverted microscope (11),
Spectrometer (9) connected to the inverted microscope (11) through a fiber-coupling optical setup (10), which is used to measure the transmission response of the plasmonic chip (2) and to determine the mass change of cells (1) by monitoring the spectral variations within the transmittance response,
The graphical user interface with algorithms, which controls the spectrometer (9) and converts its spectral output into meaningful MAR information.

The method of detecting the biophysical properties and the changes within as well as the therapeutic profiles of cells against the molecules that cause these changes with the use of biosensor platform invention in a label-free and ex vivo fashion:
Placing the cell to be examined (1) on the surface of the plasmonic chip (2), which consists of periodic nanohole array fabricated on a nm-thick metal film (16),
Placing the plasmonic chip (2), with surface where the cell (1) is attached on, to a sample holder (4) containing the cell medium (12),
Illumination of the plasmonic chip (2) with a light source (7) in the visible light spectrum,
Filtering the portion of light (13) coming into the plasmonic chip (2) by the periodic nanohole array (18), and allowing the rest to pass in a spectral window of 50 nm in the visible light spectrum,
Collecting light (14) transmitted from the plasmonic chip (2) with the objective lens of the microscope (11),
Determination of the mass accumulation profile of the whole population by measuring the mass accumulation behavior of individual cells on the surface of the plasmonic chip (2) with an optical read-out device consecutively or simultaneously.

The cell (1) to be examined is placed on the surface of the plasmonic chip (2). The surface of the plasmonic chip is coated with a surface modification agent (3) before the incubation (seeding the cells onto the surface) so that the cells can effectively adhere onto the surface. These agents can be proteins such as collagens for adherent cells or polymers such as Poly-L-Lysine for suspension cells. For a healthy cell proliferation, the plasmonic chip (2), on which the cell (1) is attached, is placed on a sample holder (4) containing the cell medium (12).

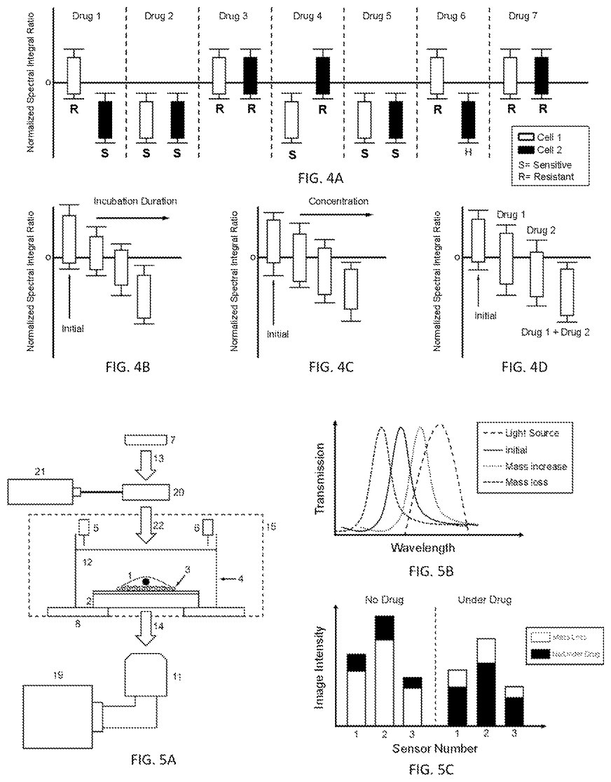

For the examination of the effects of cancer drugs, they are added to the cell medium for a certain period of time before the test. This period of time, when the cells remained in the drug-containing medium before each test, is denoted as the incubation duration in FIG. 4B. For example, if the incubation duration is 3 hours, cells are incubated in medicated media for 3 hours before testing. In addition to the incubation duration, cells are kept in the medicated medium during the drug tests.

The plasmonic chip (2) consists of periodic nanohole array (18) fabricated on a nm-thick metal film (16) (a thickness between 100 and 150 nm) (FIG. 1B). Periodic nanohole array is a periodic structure composed of circular holes with a diameter smaller than the wavelength of the light source used in the test. For example, for visible light spectroscopy (Light spectrum: 380-750 nm, diameter of circular holes: 200 nm) metal film (Example: gold or aluminum) stands on a glass substrate which is thick enough to provide strong support to the metal film, and transparent such that it does not block light transmission (17).

The plasmonic chip (2) is illuminated with a broadband light source (7) (Example: halogen lamp or white light emitting diode [LED]). While some of the light (13) reaching the plasmonic chip (2) is filtered by the periodic nanohole array (18), it is allowed to pass at certain wavelengths. The filtering region of the plasmonic chip depends on the periodicity of the nanohole array. Example: For a gold plasmonic chip with nanohole array period of 600 nm, the filtering region is located at 650 nm. In other words, the transmission response of the plasmonic chip is maximized at 650 nm.

The light (14) transmitted from the plasmonic chip (2) is collected with the objective lens of the microscope (11), and transmitted to the spectrometer (9) with a fiber-coupling optical setup (10) while its amplitude is measured for each wavelength to determine the transmission response of the plasmonic chip (FIG. 1C).

Uptake or secretion of molecular contents plays an important role in cell proliferation. Net biomass increases over time as the number of molecules accumulated is greater than the number of molecules secreted (FIG. 2A: the cell accumulates mass). Net biomass decreases over time as the number of molecules accumulated is smaller than the number of molecules secreted (FIG. 2D: the cell loses mass).

Real-time mass accumulation shifts the transmission response of the plasmonic chip (2) to longer wavelengths (FIG. 2B). Real-time mass loss shifts the transmission response of the plasmonic chip (2) to shorter wavelengths (FIG. 2E).

The cell behavior measured by the invention is determined by real-time monitoring of the transmission response of the plasmonic chip (2). Spectral changes are determined by calculating the integral of the transmission response in the integral region shown in FIG. 2B and FIG. 2E.

MAR profiling of a population is performed by real-time testing the cells of the population on the same plasmonic chip (2) surface. As the mass of each cell is different from each other, transmission response of the nanohole array is positioned at different wavelengths by upto 1 nm from each other. Therefore, the spectral integral region is positioned at 2 nm longer compared to the transmission response of the nanohole array. Bandwidth of the integral region is 60 nm.

As the cell mass on the surface of the plasmonic chip (2) increases, the transmission response of the plasmonic chip shifts to longer wavelengths (FIG. 2B). As the transmission response shifts to longer wavelengths, larger values within the transmission response overlapping with the integral region increases such that the integral value increases with time. Spectral integral value is calculated for the transmission response measured at different time periods (Example: t0, t1, t2, t3) (FIG. 2C). A linear curve for spectral integral-time data is determined as shown in FIG. 2C, and the slope of this linear curve is called mass accumulation rate (MAR). As the integral value increases for mass accumulation with time, the slope of the linear curve is calculated as a positive number. In other words, MAR is a positive value for a proliferating cell.

On the other hand, for a cell with mass decreasing with time, the transmission response shifts to shorter wavelengths (FIG. 2E) and the amount of larger values within the transmission response overlapping with the integral region decreases, which decreases the integral value with time (FIG. 2F). Spectral integral value is calculated for the transmission responses measured in different time periods (Example: t0, t1, t2, t3). As the integral values decrease with time, slope of the linear curve is calculated as a negative number as shown in FIG. 2F.

In the invention, the analog of cell mass is the spectral integral. Cells with large mass shift the transmission response of the plasmonic chip (3) more compared to cells with smaller mass.

The linear relationship between spectral integral and time is called spectral integral ratio. In the invention, the analog of MAR is the spectral integral ratio. As a result, for a cell losing mass (indicating cell death), MAR is a negative number.

Profiling cellular mass accumulation, the invention determines the biophysical properties of cells, and the therapeutic profile of cancer cells. For example, an intracellular pathway is revealed by examining the cells with the invention under an external factor stimulating this pathway. In addition, the change within the mass of cells exposed to cancer drugs is used to determine the therapeutic effects of drugs on cells.

With the invention, MAR profile of each cell in a population is determined. FIG. 3A shows two cells with positive (accumulating mass) and negative (losing mass) MAR. MAR values calculated for each cell are then mapped on the mass of these cells to determine the mass accumulation profile (MAR vs. mass plot) of the population (FIG. 3B). In this map, the value corresponding to the cell mass is the first spectral integral value of data collected during the test for each cell (For example, in FIG. 3A: initial mass of Cell† is m†, while initial mass of Cell‡ is m‡. m‡>m†). FIG. 3B shows the locations of these two cells on the MAR map, where there MAR values were calculated in FIG. 3A.

From the 2-dimensional MAR—mass (in other words, spectral integral ratio vs. spectral integral) data, the 1-dimensional normalized MAR profile (in other words, normalized spectral integral ratio) is obtained by dividing each cell's own MAR by its own mass (FIG. 3C). FIG. 3C shows the locations of the two cells, with MAR calculated in FIG. 3A, on the normalized MAR map. The normalization process eliminates the mass-dependent MAR behavior, revealing the accurate MAR profile of the cells.

Normalized MAR profile is used to determine mass accumulation and therapeutic profiles of cells.

For cells sensitive to a drug therapy, this cancer drug causes cell death. Cells undergoing apoptosis decrease in mass such that the transmission response of the plasmonic chip (2) shifts towards shorter wavelengths relative to its initial spectral position, and the calculated spectral integral value decreases. In contrast, cells resistant to the same drug treatment proliferate normally under the drug therapy such that the transmission response of the plasmonic chip (2) shifts towards longer wavelengths relative to its initial spectral position, and the calculated integral value increases.

As shown in FIG. 4A, the invention determines the responses of various cell models to different drug therapies. MAR profiles of cells sensitive drug therapies are negative, while cells resistant to drug therapies possess the same profile when they are in normal conditions.

With the invention, calibration studies can be performed for cancer drugs.

In FIG. 4B, therapeutic profile of a population is determined for a drug therapy (for a cell model sensitive to this therapy) at different incubation times. Here, as the incubation time increases, MAR decreases due to the greater loss of cell mass.

In FIG. 4C, therapeutic behavior of cells at different concentrations of a drug (for a cell model sensitive to this therapy) is shown. As the concentration increases, MAR decreases as cells lose larger mass. Characterization study with the drug concentration determines the minimum detectable concentration with the invention.

With the invention, options for the drug combination therapy can be evaluated. As an example, FIG. 4D shows a two-drug therapy results for a cell model sensitive to both of the drugs. Since two-drug therapy is more effective compared to single-drug therapies, the decrease in MAR for two-drug therapy is larger than single-drug therapies.

By dividing the working wavelength range of the spectrometer used in the invention with more than one optical grating, its spectral resolution is reduced below 1 Angstrom. Possessing high spectral resolution, MAR profile of cells is determined within short time intervals (within the order of minutes). Cell masses show small changes within the order of 0-1 picogram/hour, i.e., they create small spectral changes (below 1 nm). The high spectral resolution of the system is able to measure these minute spectral changes.

An accurate MAR profile data is determined by the system based on a spectrometer. Despite its high sensitivity, in this system, each sensor is measured sequentially, which prolongs the measurement duration so that it limits throughput (1 cell measurement in 10 minutes). Adding a camera (CCD or CMOS) within the operating range of the spectrometer and a narrow-band light source (0 to 5 nm) to the system, throughput could be dramatically increased (FIG. 5A).

In the invention, after removing the spectrometer (9) and the fiber-coupling optical setup (10) allowing light transmission to the spectrometer (9), the two parts are integrated (FIG. 5A):
LC (liquid crystal) filter (20) assembled on the light source (7),
Camera (19) reading the transmission response of the plasmonic chip (2).

The invention shown in FIG. 5A is another version of the plasmonic-based biosensor platform which contains:
Plasmonic chip (2), which consists of periodic nanohole array (18) fabricated on a nm-thick metal film (16), with surface seeded with cells to be examined,
Light source (7) that illuminates the plasmonic chip (2),
Inverted microscope (11), which is used to illuminate the plasmonic chip (2), to collect the light transmitted from the chip, and to send it to the camera (19) for optical read-out,
Incubator case (15) that provides the incubator conditions for cell culture (Example: 5% CO₂, 37% Temperature, 95% Humidity), and contains CO₂ module (5), humidity module (6) and heat module (8) integrated into the inverted microscope (11),
Camera (19), which determines the spectral variations within the transmission response of the plasmonic chip (2) due to mass changes of cells (1) via monitoring the changes within the light intensity,
Light source (7) for the illumination of the plasmonic chip (2) and LC filter (20) assembled on the source,
Graphical user interface with algorithms controlling the camera (19) and LC filter (20), and converting the light intensity data into meaningful MAR information.

Filtering range of the LC filter (20) is controlled by the LC control unit (21). As shown in FIG. 5B, light transmitted from the filter (22) is spectrally positioned at longer wavelengths compared to the transmission response of the plasmonic chip.

For a cell with mass increasing with time, transmission response of the plasmonic chip (2) shifts to longer wavelengths, and spectrally better overlaps with the light source generated by the LC filter (20). Thus, more photons pass through the plasmonic chip (2) such that the image intensity of the transmitted light (14) measured with the camera (19) increases.

On the other hand, for a cell with mass decreasing with time, transmission response of the plasmonic chip (2) shifts toward shorter wavelengths, and the image intensity of the transmitted light (14) from the plasmonic chip (2) and measured with the camera (19) decreases.

Here, the filtering window of the LC filter is critical for high-precision determination of spectral changes with the system. Detection sensitivity of the system is determined by the bandwidth of the filter. Narrower the LC filter bandwidth, spectral changes within the transmission response of the nanohole array due to the accumulation or loss of cell mass on the sensor surface create more contrast in the camera.

Cells incubated on different sensor locations on the plasmonic chip (2) surface (a single cell locates in each sensor region) are monitored simultaneously to determine the change in their mass. The changes within the cell mass are then used to determine the therapeutic profile of cells exposed to cancer drugs.

In FIG. 5C, the sensor regions are enumerated as 1, 2, and 3. For an heathy proliferating cell (in the absence of a drug), an increase in mass (positive MAR) is observed as an increase in the image intensity taken by the camera. For cells in a medium containing a drug that are sensitive to this drug, a decrease in mass (negative MAR) is observed as a decrease in the image intensity.

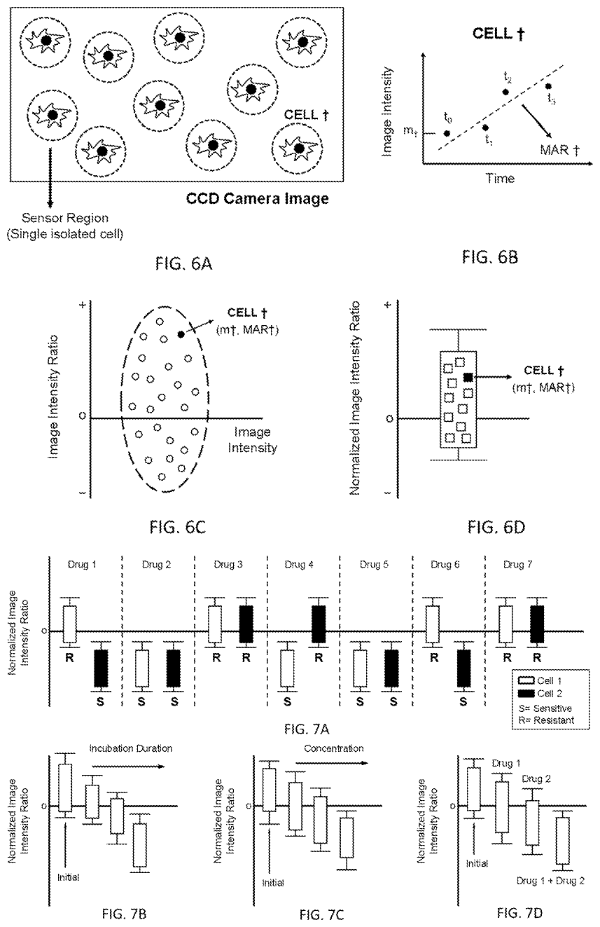

In the high-throughput version of the invention, cells are automatically selected with a graphical user interface as shown in FIG. 6A, and the image intensities are monitored within these regions. The graphical user interface determines the borders of the cells in the camera images, and uses the camera (19) pixels in the regions bordered with the cell membrane for MAR analyses. By obtaining real-time light intensity data from a region possessing no cell and subtracting this data from the one obtained for the sensor regions, minimize the noise due to the background signal. In order to have accurate MAR analyses for population studies, cells close to mitosis are eliminated as they have about twice the size of a normal cell and have distinct characteristics compared to the general behavior of the population.

In the camera-integrated invention, the analog of cell mass is image intensity. In the camera (19), cells with larger mass increase the image intensity of the plasmonic chip (2) more compared to cells with smaller mass. MAR profile is calculated from the image intensity ratio, which is the slope of the linear relationship between image intensity and time (FIG. 6B). In FIG. 6B, MAR is calculated as a positive value for a cell increasing mass by molecular uptake (image intensity increases).

Using the calculated mass and MAR values for each cell, MAR vs. cell mass map is generated to reveal the MAR profile of the population (FIG. 6C). Here, the mass of each cell corresponds to the initial value of the image intensity determined in the beginnings of each MAR test (For example, in FIG. 6B: initial mass of Cell† is m†). FIG. 6C shows the location of the cell with MAR calculated in FIG. 6B on the MAR map.

From the 2-dimensional MAR—mass (in other words, image intensity ratio vs. image intensity) data, the 1-dimensional normalized MAR profile (in other words, normalized image intensity ratio) is determined by dividing the MAR value calculated for each cell by its own mass (FIG. 6D). FIG. 6D shows the location of the cell with MAR calculated in FIG. 6B on the normalized MAR map. Normalization process eliminates the mass-dependent MAR behavior, revealing the accurate MAR profile of the cells.

Normalized MAR profile is used to determine the mass accumulation and therapeutic profiles of cells.

For cells sensitive to a drug therapy, this cancer drug causes cell death. Cells undergoing apoptosis decrease in mass, which reduces the image intensity of the plasmonic chip (2) taken by the camera (19). In contrast, cells resistant to the same drug treatment proliferate normally under the drug therapy such that the image intensity of the plasmonic chip (2) in the camera (19) increases.

As shown in FIG. 7A, the invention determines the responses of various cell models to different drug therapies. MAR profiles of cells sensitive drug therapies are negative, while cells resistant to drug therapies possess the same profile when they are in normal conditions.

With the invention, calibration studies can be performed for cancer drugs.

In FIG. 7B, therapeutic profile of a population is determined for a drug therapy (for a cell model sensitive to this therapy) at different incubation times. Here, as the incubation time increases, MAR decreases due to the greater loss of cell mass.

In FIG. 7C, therapeutic behavior of cells at different concentrations of a drug (for a cell model sensitive to this therapy) is shown. As the concentration increases, MAR decreases as cells lose larger mass. Characterization study with the drug concentration determines the minimum detectable concentration with the invention.

With the invention, options for the drug combination therapy can be evaluated. As an example, FIG. 7D shows a two-drug therapy results for a cell model sensitive to both of the drugs. Since two-drug therapy is more effective compared to single-drug therapies, the decrease in MAR for two-drug therapy is larger than single-drug therapies.

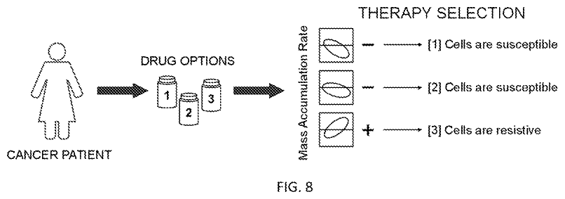

Cancer cells taken from patients with biopsy are loaded to the invention. MAR profiles of cells exposed to different drugs are revealed with the system. For example, as shown in FIG. 8, single drug or drug combination treatments that negatively affects MAR is determined for a diagnosed cancer type, and the physician uses these drugs in therapy. An unvarying MAR profile indicates that the cancer type under investigation is resistant to the tested drug therapies. Avoiding the wrong drug therapy options by the physician, incorrect treatments resulting in loss of time and increasing health cost is prevented.

### REFERENCES

Cermak, N., Olcum, S., Delgado, F. F., Wasserman, S. C., Payer, K. R., Murakami, M., Knudsen, S. M., Kimmerling, R. J., Stevens, M. M., Kikuchi, Y., Sandikci, A., Ogawa, M., Agache, V., Baleras, F., Weinstock, D. M., Manalis, S. R. 2016. “High-throughput single-cell growth measurements via serial microfluidic mass sensor arrays”, Nature Biotechnology, 34, 1052-1059
Eletxigerra, Martinez-Perdiguero, J., Barderas, R., Pingarron, J. M., Campuzano, S., Merino, S. 2016. “Surface plasmon resonance immunosensor for ErbB2 breast cancer biomarker determination in human serum and raw cancer cell lysates”, Analytica Chimica Acta, 905, 156e162.
Elfwing, A., LeMarc, Y., Baranyi, J., Ballagi, A. 2004. “Observing Growth and Division of Large Numbers of Individual
Giner-Casares, J. J., Henriksen-Lacey, M., Garcia, I., Liz-Marz, L. M. 2016. “Plasmonic surfaces for cell growth and retrieval triggered by near-infrared light”, Angewandte Chemie International Edition, 55, 974-78.
Haibe-Kains, B., El-Hachem, N., Birkbak, N. J., Jin, A. C., Beck, A. H., Aerts, H. J. W. L., Quackenbush, J. 2013. “Inconsistency in large pharmacogenomic studies”, Nature, 504, 389-93.
Kim, K., Kim, S., Jeon, J. S. 2018. “Visual estimation of bacterial growth level in microfluidic culture systems”, Sensors, 18, 447.
Li, X., Soler, M., Ozdemir, C. I., Belushn, A., Yesilkoy, F., Altug, H. 2017. “Plasmonic nanohole array biosensor for label-free and real-time analysis of live cell secretion”, Lab on a Chip, 17, 2208-2217
Li, X., Soler, M., Szydzik, C., Khoshmanesh, K., Schmidt, J., Coukos, G., Mitchell, A., Altug, H. 2018. “Label-free optofluidic nanobiosensor enables real-time analysis of single-cell cytokine secretion”, Small, 14, e1800698.
Massad-Ivanir, N., Shtenberg, G., Segal, E. 2013. “Optical detection of E. coli bacteria by mesoporous silicon biosensors”, Journal of Visualized Experiments: JoVE, 81, 50805.
Masson, J. F., Zhao, S. S. 2015. “Plasmonic sensors for analysis of proteins and an oncologic drug in human serum”. Nanobiosensors and Nanobioanalyses. Editörler: Vestergaard, M., Kerman, K., Hsing, I.M., Tamiya, E. Tokyo: Springer
Mellinghoff, I. K., Wang, M. Y., Vivanco, I., Haas-Kogan, D. A., Zhu, S., Dia, E. Q., Lu, K. V., Yoshimoto, K., Huang, J. H., Chute, D. J., Riggs, B. L., Horvath, S., Liau, L. M., Cavenee, W. K., Rao, P. N., Beroukhim, R., Peck, T. C., Lee, J. C., Sellers, W. R., Stokoe, D., Prados, M., Cloughesy, T. F., Sawyers, C. L., Mischel, P. S. 2005. “Molecular determinants of the response of glioblastomas to EGFR kinase inhibitors”, The New England Journal of Medicine, 353, 2012-24.
Stevens, M. M., Maire, C. L., Chou, N., Murakami, M., Knoff, D., Kikuchi, Y., Kimmerling, R. J., Liu, H., Haidar, S., Calistri, N. L., Cermak, N., Olcum, S., Cordero, N., Idbaih, A., Wen, P. Y., Weinstock, D. M., Ligon, K. L., Manalis, S. R. 2016. “Drug sensitivity of single cancer cells is predicted by changes in mass accumulation rate”, Nature Biotechnology, 11, 1161-1167.
Tu, L., Li, X., Bian, S., Yu, Y., Li, J., Huang, L., Liu, P., Wu, Q., Wang, W. 2017. “Label-free and real-time monitoring of single cell attachment on template-stripped plasmonic nanoholes”, Scientific Reports, 7, 11020.
Van Der Hofstadt, M., fliittener, M., Juárez, A., Gomila, G. 2015. “Nanoscale imaging of the growth and division of bacterial cells on planar substrates with the atomic force microscope”, Ultramicroscopy, 154, 29-36.

Zhang, F., Wang, S., Yin, L., Yang, Y., Guan, Y., Wang, W., Xu, H., Tao, N. 2015. “Quantification of epidermal growth factor receptor expression level and binding kinetics on cell surfaces by surface plasmon resonance imaging”, Analytical Chemistry, 87, 9960-65.

What is claimed is:

1. A plasmonic-based biosensor platform for detecting biophysical properties of cells and changes within, as well as a therapeutic response of the cells against molecules causing the changes in a label-free and ex vivo fashion, comprising:
a plasmonic chip consisting of a periodic nanohole array fabricated on a nm-thick metal film and a surface where the cells to be examined are seeded,
a light source illuminating the plasmonic chip,
an inverted microscope used to illuminate the plasmonic chip to collect a light transmitted from the plasmonic chip and to send the light to an optical read-out device,
an incubator case providing incubator conditions for a cell culture and integrated to the inverted microscope,
the optical read-out device measuring a transmission response of the plasmonic chip and integrated to the inverted microscope,
a graphical user interface with algorithms controlling the optical read-out device and converting outputs of the optical read-out device into meaningful MAR information.

2. The plasmonic-based biosensor platform according to claim 1, wherein the optical read-out device is a spectrometer coupled to the inverted microscope with a fiber coupling-optical setup.

3. The plasmonic-based biosensor platform according to claim 1, wherein the optical read-out device is a camera.

4. The plasmonic-based biosensor platform according to claim 3, wherein the optical read-out device comprises a liquid crystal (LC) filter assembled on the light source when the camera is present.

5. The plasmonic-based biosensor platform according to claim 4, wherein the LC filter is in a bandwidth range of 0-5 nm.

6. The plasmonic-based biosensor platform according to claim 1, wherein the plasmonic-based biosensor platform has an ability to determine a cell mass and to detect real-time changes within.

7. The plasmonic-based biosensor platform according to claim 1, wherein the plasmonic-based biosensor platform has an ability to determine a mass accumulation behavior and the therapeutic response of single cells or cell populations.

8. The plasmonic-based biosensor platform according to claim 7, wherein the plasmonic-based biosensor platform has an ability to determine therapeutic effects of cancer drugs on cancer cells in a real-time, label-free and ex vivo fashion.

9. The plasmonic-based biosensor platform according to claim 1, wherein a sensitivity of the plasmonic-based biosensor platform is within a range of 0-1 picogram/hour.

10. A device comprising the plasmonic-based biosensor platform according to claim 1.

11. A method of detecting the biophysical properties of the cells, biophysical changes of the cells, and a therapeutic behavior of the cells against molecules causing the biophysical changes in the label-free and ex vivo fashion using the plasmonic-based biosensor platform according to claim 1, comprising a determination of changes in a mass of cells seeded on the surface of the plasmonic chip, wherein single cells are positioned in each sensor region, with a use of spectral changes within the transmission response of the periodic nanohole array or light intensity changes in a single cell level.

12. A method of determining the biophysical properties, the changes within and a therapeutic behavior of the cells against the molecules causing changes in the label-free and ex vivo fashion with the plasmonic-based biosensor platform according to claim 1, comprising steps of:
seeding the cells to be examined on the surface of the plasmonic chip consisting of the periodic nanohole array fabricated on the nm-thick metal film,
placing the plasmonic chip with the surface where the cells are seeded on in a sample holder containing a cell medium,
illuminating the plasmonic chip with the light source in a visible light spectrum,
filtering some light coming into the plasmonic chip by the periodic nanohole array, and allowing a filtered light to pass in a spectral window of 50 nm within the visible light spectrum,
collecting the light transmitted from the plasmonic chip with an objective lens of the inverted microscope,
determining a of the cells by measuring a mass accumulation behavior of single cells on the surface of the plasmonic chip with the optical read-out device consecutively or simultaneously.
